(19) United States
(12) Patent Application Publication
(10) Pub. No.: US 2013/0190424 A1
(43) Pub. Date: Jul. 25, 2013

(54) MELAMINE EPOXY RESIN MONOMER AND RESIN COMPOSITION

(71) Applicants: Hiroaki Takamatsu, Namerikawa-shi (JP); Kyoko Yamamoto, Namerikawa-shi (JP); Hideo Higashi, Namerikawa-shi (JP)

(72) Inventors: Hiroaki Takamatsu, Namerikawa-shi (JP); Kyoko Yamamoto, Namerikawa-shi (JP); Hideo Higashi, Namerikawa-shi (JP)

(73) Assignee: NIPPON CARBIDE INDUSTRIES CO., INC., Minato-ku, Tokyo (JP)

(21) Appl. No.: 13/823,140

(22) PCT Filed: Sep. 14, 2011
(86) PCT No.: PCT/JP2011/071042

§ 371 Date: Apr. 5, 2013

(30) Foreign Application Priority Data
Sep. 29, 2010 (JP) 2010-219953

(51) Int. Cl. C08L 79/04; C07D 407/02

(52) U.S. Cl. CPC C08L 79/04; C07D 407/02
USPC 523/400; 544/198; 252/182.23

(57) ABSTRACT

A melamine epoxy resin monomer including: a glycidyl group; and a structural unit having a melamine residue and being represented by the following Formula (I) is disclosed. In Formula (I), each of R¹ to R⁴ independently represents a hydrogen atom, a group represented by R⁵OCH₂—, or a group derived from a melamine derivative and represented by the following Formula (II). R⁵ represents a hydrogen atom, an alkyl group having 1 to 4 carbon atoms, or a glycidyl group. In Formula (II), each of R²¹ to R²⁵ independently represents a hydrogen atom, a group represented by R²⁶OCH₂—, or a group derived from a melamine derivative and represented by Formula (II). R²⁶ represents a hydrogen atom, an alkyl group having 1 to 4 carbon atoms, or a glycidyl group.

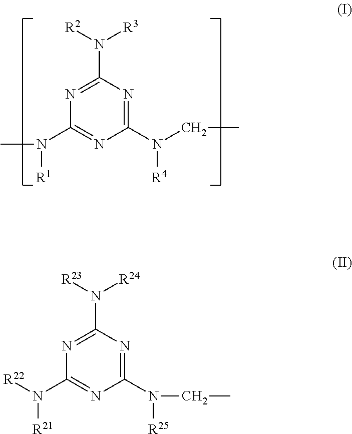

### CROSS-REFERENCE TO RELATED APPLICATION

This application is the National Stage of International Application No. PCT/JP2011/071042, filed Sep. 14, 2011, which claims the benefit of and priority to JP 2010-219953, filed Sep. 29, 2010, the contents of which are incorporated by reference as if fully set forth herein.

### TECHNICAL FIELD

The present invention relates to melamine epoxy resin monomers and resin compositions.

### BACKGROUND ART

Reliability demanded from sealants in semiconductor/electronic instrument devices has been increasing in accordance with higher outputs as well as slimming down and miniaturization of the devices. For example, optical semiconductor elements such as LEDs and LDs (laser diodes) are small and efficiently emit light with vivid colors. Also, such elements have long lives, excellent drive performance, and high durability against vibration and the repetition of switching ON/OFF because of being semiconductor elements. Therefore, the elements are utilized as various indicators and various light sources.

As one of package materials in which such optical semiconductor elements such as LEDs are used, a polyphthalamide resin (PPA), which is a colorless or white material, is currently widely used.

However, the higher outputs and shorter wavelengths of optical semiconductor devices have been remarkably achieved due to the current-day rapid progress of optical semiconductor technologies. Therefore, in optical semiconductor devices such as photocouplers which are capable of emitting or receiving high-energy light, conventional semiconductor element sealants and cases in which PPA resins are used undergo remarkable deterioration due to long-term use, and coloring of packages, generation of color uneveness, removal of sealing resin, reduction in mechanical strength, and the like are prone to occur. Therefore, it is desired to effectively solve such problems.

In relation to the above, Japanese Patent Publication No. 7-22943 proposes a pre-molded package containing polyester and silicone and describes that the pre-molded package is excellent in heat resistance and adhesiveness. In addition, Japanese Patent Application Laid-Open (JP-A) No. 2002-302533 proposes an epoxy resin composition for sealing an optical semiconductor containing an intermediate reactant of an epoxy resin and a curing agent and describes that the epoxy resin composition is excellent in transparency and solder resistance. Further, Japanese Patent Application Laid-Open (JP-A) No. 2010-31269 proposes a silicone resin-epoxy resin composition and describes that a cured product excellent in heat resistance and light resistance is obtained.

### DISCLOSURE OF INVENTION

### Technical Problem

However, there are cases in which it is difficult to say that even such resin compositions as described above have sufficient performance in terms of light resistance and an optical reflectance.

An object of the present invention is to provide a resin composition capable of forming a cured product having excellent light resistance and a high optical reflectance; and a melamine epoxy resin monomer suitable for the resin composition.

### Solution to Problem

The present invention encompasses the following embodiments:

<1> A melamine epoxy resin monomer comprising: a glycidyl group; and a structural unit having a melamine residue and being represented by the following Formula (I).

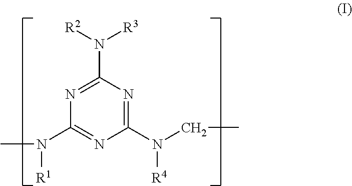

(In Formula (I), each of R¹ to R⁴ independently represents a hydrogen atom, a group represented by R⁵OCH₂—, or a group derived from a melamine derivative and represented by the following Formula (II); and R⁵ represents a hydrogen atom, an alkyl group having 1 to 4 carbon atoms, or a glycidyl group.)

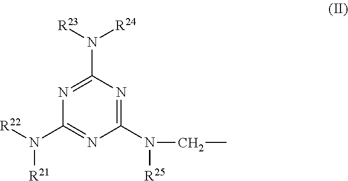

(In Formula (II), each of R²¹ to R²⁵ independently represents a hydrogen atom, a group represented by R²⁶OCH₂—, or a group derived from a melamine derivative and represented by Formula (II); and R²⁶ represents a hydrogen atom, an alkyl group having 1 to 4 carbon atoms, or a glycidyl group.)

<2> A melamine epoxy resin monomer comprising a glycidyl group and a melamine residue and being represented by the following Formula (III).

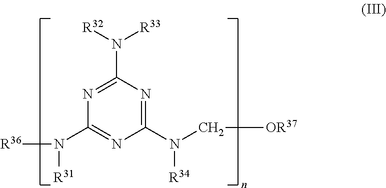

(In Formula (III), each of R³¹ to R³⁴ independently represents a hydrogen atom, a group represented by R³⁵OCH₂—, or a group derived from a melamine derivative and represented by the following Formula (II); R³⁶ represents a hydrogen atom or a group represented by R³⁸OCH₂—; each of R³⁵, R³⁷, and R³⁸ independently represents a hydrogen atom, an alkyl group having 1 to 4 carbon atoms, or a glycidyl group; and n represents an integer from 1 to 8.)

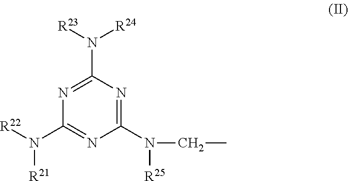

(In Formula (II), each of R²¹ to R²⁵ independently represents a hydrogen atom, a group represented by R²⁶OCH₂—, or a group derived from a melamine derivative and represented by Formula (II); and R²⁶ represents a hydrogen atom, an alkyl group having 1 to 4 carbon atoms, or a glycidyl group.)

<3> The melamine epoxy resin monomer according to <1> or <2>, wherein the number of the contained glycidyl groups is 2 or more.

<4> The melamine epoxy resin monomer according to any one of <1> to <3>, wherein the number of the contained melamine residues is 8 or less.

<5> A resin composition comprising: the melamine epoxy resin monomer according to any one of <1> to <4>; and an inorganic filler.

<6> The resin composition according to <5>, further comprising a curing agent.

<7> A composition for light reflection, which is a cured product of the resin composition according to <5> or <6>.

### Effects of Invention

In accordance with the present invention, a resin composition capable of forming a cured product having excellent light resistance and a high optical reflectance, and a melamine epoxy resin monomer suitable for the resin composition can be provided.

### BRIEF DESCRIPTION OF DRAWINGS

FIG. 1 is a view that indicates an example of the ¹H-NMR spectrum of the melamine epoxy resin monomer according to the present invention.

FIG. 2 is a view that indicates an example of the FT-IR spectrum of the melamine epoxy resin monomer according to the present invention.

FIG. 3 is a view that indicates an example of the ¹H-NMR spectrum of the melamine epoxy resin monomer according to the present invention.

FIG. 4 is a view that indicates an example of the FT-IR spectrum of the melamine epoxy resin monomer according to the present invention.

### BEST MODE FOR CARRYING OUT THE INVENTION

The term “step” as used herein encompasses not only an independent step but also a step, in which the anticipated effect of the step is achieved, even if the step cannot be definitely distinguished from another step. In addition, a numerical value range indicated by using “ . . . to . . . ” as used herein refers to a range including numerical values described before and after the “to” as the minimum and maximum values, respectively. Further, for mentioning the amount of each component in a composition in the present specification, when plural substances corresponding to each component are present in the composition, the amount of each component in the composition means the total amount of the plural substances present in the composition unless otherwise specified.

### <Melamine Epoxy Resin Monomer>

The melamine epoxy resin monomer according to the present invention is characterized by including at least one structural unit having a melamine residue and represented by the following Formula (I); and a glycidyl group. The melamine epoxy resin monomer having such a particular structure is excellent in light resistance and, for example, can suppress occurrence of yellowing due to light irradiation, and can maintain a high optical reflectance for a long time.

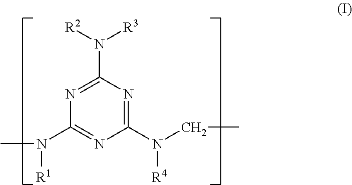

In Formula (I), R¹ to R⁴ each of R¹ to R⁴ independently represents a hydrogen atom, a group represented by R⁵OCH₂— (hereinafter also simply referred to as “R⁵OCH₂-”), or a group derived from a melamine derivative and represented by the following Formula (II).

In addition, R⁵ represents a hydrogen atom, an alkyl group having 1 to 4 carbon atoms, or a glycidyl group.

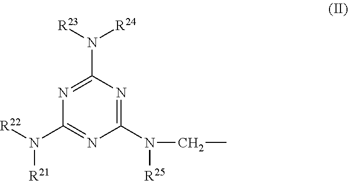

In Formula (II), each of R²¹ to R²⁵ independently represents a hydrogen atom, a group represented by R²⁶OCH₂—, or a group derived from a melamine derivative and represented by Formula (II). R²⁶ represents a hydrogen atom, an alkyl group having 1 to 4 carbon atoms, or a glycidyl group.

In view of light resistance, it is preferable that R¹ to R⁴ in Formula (I) represent R⁵OCH₂— or a group derived from a melamine derivative and represented by Formula (II) in view of it is more preferable that at least one of R¹ to R⁴ represents R⁵OCH₂—, and it is further preferable that at least three of R¹ to R⁴ represent R⁵OCH₂—.

R⁵ represents a hydrogen atom, an alkyl group having 1 to 4 carbon atoms, or a glycidyl group, and preferably represents an alkyl group having 1 to 4 carbon atoms or a glycidyl group in view of light resistance. When two or more R⁵s are included in the structural unit represented by Formula (I), each of the two or more R⁵s may be the same or different. Further, when two or more R⁵s are included in the structural unit represented by Formula (I), at least one of the two or more R⁵s is preferably a glycidyl group.

In the melamine epoxy resin monomer, it is preferable in view of light resistance that at least three of R¹ to R⁴ in Formula (I) represent R⁵OCH₂—, at least one of types of R⁵ represent a glycidyl group, and at least one of remaining R⁵ represent an alkyl group having 1 to 4 carbon atoms.

When the melamine epoxy resin monomer includes two or more structural units represented by Formula (I), R¹ to R⁴ in each structural unit may be respectively the same or different.

In the group derived from the melamine derivative represented by Formula (II), each of R²¹ to R²⁵ independently represents a hydrogen atom, R²⁶OCH₂—, or a group derived from a melamine derivative and represented by Formula (II). In the present invention, in view of light resistance, it is preferable that R²¹ to R²⁵ represent R²⁶OCH₂— or a group derived from a melamine derivative and represented by Formula (II), it is more preferable that at least one of R²¹ to R²⁵ represents R²⁶OCH₂—, and it is further preferable that at least three of R²¹ to R²⁵ represent R²⁶OCH₂—.

R²⁶ represents a hydrogen atom, an alkyl group having 1 to 4 carbon atoms, or a glycidyl group, and preferably represents an alkyl group having 1 to 4 carbon atoms or a glycidyl group in view of light resistance. When two or more R²⁶s are included in the group derived from the melamine derivative represented by Formula (II), the two or more R²⁶s may be respectively the same or different. Further, when two or more R²⁶s are included in the group derived from the melamine derivative represented by Formula (II), at least one of the two or more R²⁶s is preferably a glycidyl group.

When the melamine epoxy resin monomer includes two or more groups derived from a melamine derivative represented by Formula (II), R²¹ to R²⁵ in each group derived from the melamine derivative may be respectively the same or different.

The melamine epoxy resin monomer includes at least one structural unit represented by Formula (I) and is preferably a compound represented by the following Formula (III) in view of light resistance and heat resistance.

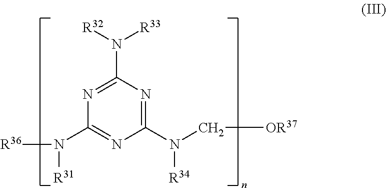

In Formula (III), each of R³¹ to R³⁴ independently represents a hydrogen atom, a group represented by R³⁵OCH₂—, or a group derived from a melamine derivative and represented by the following Formula (II). R³⁶ represents a hydrogen atom or a group represented by R³⁸OCH₂—. each of R³⁵, R³⁷, and R³⁸ independently represents a hydrogen atom, an alkyl group having 1 to 4 carbon atoms, or a glycidyl group. n represents an integer from 1 to 8.

R³¹ to R³⁵ in Formula (III) have the same definitions as R¹ to R⁵ in Formula (I) and the preferable embodiments thereof are also the same.

Further, in Formula (III), the group derived from the melamine derivative represented by Formula (II) has the same definitions as the group derived from the melamine derivative represented by Formula (II) in the structural unit represented by Formula (I) and the preferable embodiments thereof are also the same.

R³⁶ represents a hydrogen atom or a group represented by R³⁸OCH₂—. In the present invention, the group represented by R³⁸OCH₂— is preferable in view of light resistance.

each of R³⁷ and R³⁸ independently represents a hydrogen atom, an alkyl group having 1 to 4 carbon atoms, or a glycidyl group. In the present invention, the alkyl group having 1 to 4 carbon atoms or the glycidyl group is preferable in view of light resistance.

n represents an integer from 1 to 8, and n is preferably an integer from 1 to 6, and more preferably from 1 to 4, in view of light resistance, heat resistance, and curability.

The number of glycidyl groups contained in the melamine epoxy resin monomer is not particularly limited. From the viewpoints of light resistance, heat resistance, and curability, the number is preferably 2 or more, and more preferably from 2 to 6.

When the melamine epoxy resin monomer is a mixture of two or more melamine epoxy resin monomers, the number of the contained glycidyl groups means the average value of the number of the glycidyl groups contained in the two or more melamine epoxy resin monomers.

The number of melamine residues contained in the melamine epoxy resin monomer is not particularly limited. In view of light resistance, heat resistance, and processing suitability, the number is preferably 8 or less, more preferably from 1 to 6, and further preferably from 1 to 4.

The number of melamine residues contained in the melamine epoxy resin monomer means the total number of melamine residues contained in structural units represented by Formula (I) or in the group derived from a melamine derivative represented by Formula (II).

Further, when the melamine epoxy resin monomer is a mixture of two or more melamine epoxy resin monomers, the number of the contained melamine residues means the average value of the number of the melamine residues contained in the two or more melamine epoxy resin monomers.

Specific examples of the melamine epoxy resin monomer contained in a structural unit represented by Formula (I) will be indicated below, but the present invention is not limited thereto. “n” in the following specific examples represents an integer from 1 to 4.

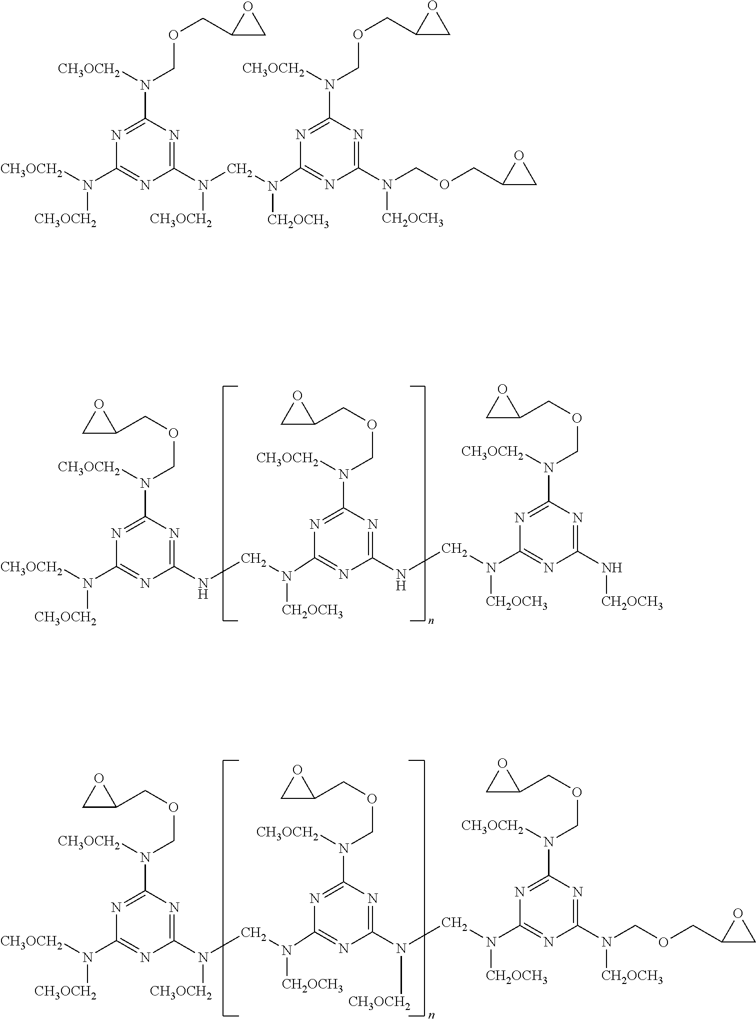

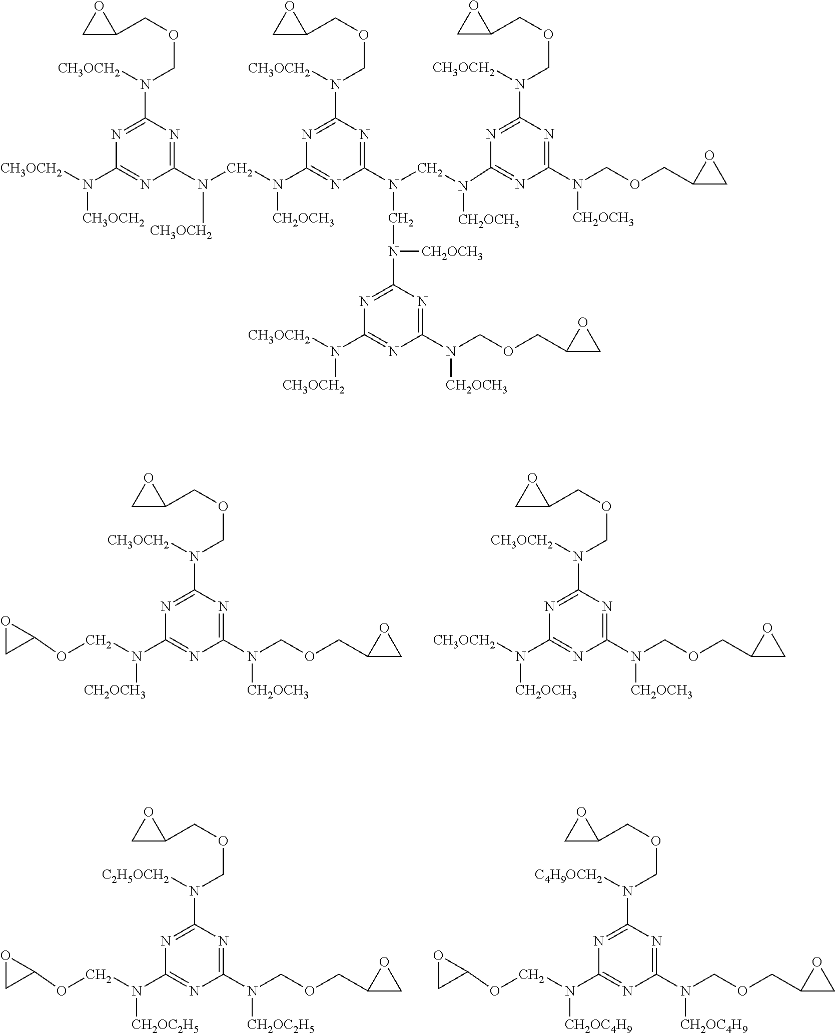

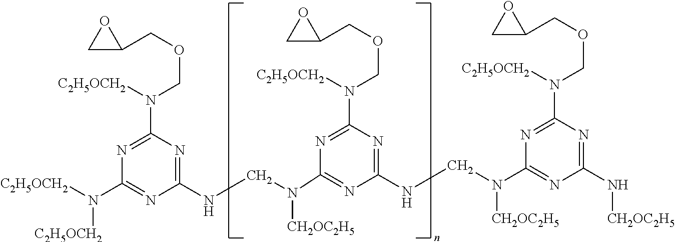

### <Method of Producing Melamine Epoxy Resin Monomer>

The melamine epoxy resin monomer can be produced by a usually used method of example, the melamine epoxy resin monomer can be produced by reacting methylol melamine obtained from melamine and an aldehyde compound (preferably, formaldehyde) with an epihalohydrin. Specifically, it is preferable to produce the melamine epoxy resin monomer by a production method such as that described below.

For example, a method of producing the melamine epoxy resin monomer is configured by including the steps of: preparing hexahydroxyalkyl melamine which is an aldehyde adduct of melamine; and reacting the hexahydroxyalkyl melamine with an epihalohydrin to introduce a glycidyl group into the hexahydroxyalkyl melamine, and by including another step as necessary.

The step of preparing the hexahydroxyalkyl melamine may be a step of reacting melamine with an aldehyde compound (preferably, formaldehyde) to produce desired hexahydroxyalkyl melamine or a step of selecting a desired hexahydroxyalkyl melamine from commercially available hexahydroxyalkyl melamines

The method of producing hexahydroxyalkyl melamine is not particularly limited as long as it enables to produce hexahydroxyalkyl melamine having a desired structure, and is appropriately selected from production methods which are usually performed to be performed.

In addition, examples of the commercially available hexahydroxyalkyl melamines may include NIKALAC MS-11 (manufactured by Nippon Carbide Industries Co., Inc.), NIKALAC MS-001 (manufactured by Nippon Carbide Industries Co., Inc.), MYCOAT 715 (manufactured by Nihon Cytec Industries Inc. Co., Ltd.), and the like.

In the step of introducing the glycidyl group into the hexahydroxyalkyl melamine, the prepared hexahydroxyalkyl melamine is reacted with an epihalohydrin. Examples of the epihalohydrin used may include epichlorohydrin, epibromohydrin, and the like, and epichlorohydrin is preferable. A reaction condition therefor is not particularly limited if a glycidyl group can be introduced into a hydroxyl group in hexahydroxyalkyl melamine, and can be appropriately selected from usually used reaction conditions.

For example, a glycidyl group can be introduced into a hydroxyl group in hexahydroxyalkyl melamine by heating a mixture of hexahydroxyalkyl melamine with an epihalohydrin in the presence of a base such as sodium hydroxide in an organic solvent in which hexahydroxyalkyl melamine can be dissolved. In this case, a phase transfer catalyst such as tetramethylammonium chloride may also be used.

In this step, the number of glycidyl groups introduced into the melamine epoxy resin monomer can be regulated by appropriately selecting the ratio of the amount of an epihalohydrin used to that of hexahydroxyalkyl melamine and reaction time.

The method of producing a melamine epoxy resin monomer may also further include the step of introducing an alkyl group into a hydroxyl group in hexahydroxyalkyl melamine A method of introducing an alkyl group into hexahydroxyalkyl melamine is not particularly limited, and can be appropriately selected from usually used methods. Examples thereof include a method of reacting hexahydroxyalkyl melamine with an alkyl alcohol in the presence of an acid.

The step of introducing an alkyl group into hexahydroxyalkyl melamine may be carried out before or after the step of introducing a glycidyl group into hexahydroxyalkyl melamine

### <Resin Composition>

The resin composition of the present invention is configured by including at least one of the melamine epoxy resin monomers and at least one inorganic filler and by including another component such as a curing agent as necessary. This configuration enables to form of a resin cured product having excellent light resistance and a high optical reflectance by heat curing.

The details and preferable embodiments of the melamine epoxy resin monomer contained in the resin composition are as described above. The melamine epoxy resin monomer contained in the resin composition may be used singly or in combination of two or more thereof. When the resin composition contains two or more melamine epoxy resin monomers, it is enough as long as the structures of the two or more melamine epoxy resin monomers are different from each other. Examples include ones of which the number of contained melamine residues are different from each other, ones of which the number of contained glycidyl groups are different from each other, ones containing alkoxymethyl groups different from each other, and combinations thereof.

The resin composition may further contain an epoxy resin monomer other than the melamine epoxy resin monomer according to the present invention.

### (Inorganic Filler)

The form of the inorganic filler is not particularly limited, and may be fiber, plate, or powder form.

Examples of the fibrous inorganic filler may include glass fibers, asbestos fibers, silica fibers, silica alumina fibers, alumina fibers, zirconia fibers, boron nitride fibers, boron fibers, and potassium titanate fibers.

In addition, examples of the powdery inorganic filler include silicates such as silica, quartz powders, glass beads, glass powders, calcium silicate, aluminum silicate, kaoline, talc, clay, diatomite, and wollastonite; metal oxides such as iron oxide, titanium oxide, zinc oxide, antimony trioxide, and alumina; metal carbonates such as calcium carbonate and magnesium carbonate; metal sulfates such as calcium sulfate and barium sulfate; and also ferrite, silicon carbide, silicon nitride, boron nitride, and aluminum nitride.

In addition, examples of the plate-like inorganic filler include mica, and glass flakes.

These inorganic fillers may be used singly or in combination of two or more thereof.

The color of the inorganic filler is not particularly limited, while it is preferably a white inorganic filler in view of light resistance and a high reflectance. Examples of the white inorganic filler include titanium oxide, zinc oxide, silica, quartz powders, talc, calcium carbonate, magnesium carbonate, calcium sulfate, barium sulfate, mica, and alumina.

In view of light resistance and a high reflectance, the inorganic filler is preferably a white inorganic filler, and more preferably at least one selected from the group consisting of titanium oxide, silica, and alumina.

The volume-average particle diameter of the inorganic filler is not particularly limited. In view of the moldability and flowability of the resin composition, the volume-average particle diameter is from 0.5 μm to 40 μm, particularly preferably from 1 μm to 35 μm. Further, it is also preferable to use particles having a volume-average particle diameter in a fine regions of 1 μm or less, particles in a middle particle diameter region of from 1 μm to 10 μm, and particles in a coarse region of from 10 μm to 40 μm in combination to highly fluidize the resin composition when being subjected to potting or underfilling.

The volume-average particle diameter of the inorganic filler can be performed using a laser diffraction scattering particle size distribution measuring device.

The content of the inorganic filler contained in the resin composition may be appropriately selected depending on a purpose. In view of light resistance and a high reflectance, the content in the resin composition is preferably from 97 mass % to 50 mass %, more preferably from 95 mass % to 75 mass %.

### (Curing Agent)

The resin composition preferably contains at least one curing agent. The curing agent is not particularly limited as long as it enables to react with an epoxy resin to form a cured product, and can be appropriately selected and used from usually used curing agents. Examples which can be used include novolac phenol resins obtained by condensation reaction of formaldehyde with acid anhydride, phenol, cresol, xylenol, resorcin, or the like, polymercapto resins such as liquid polymercaptan and polysulphide, and amide- and amine-based curing agents, as well as acrylates, carbonates, isocyanates, and the like. Among them, non-aromatic compound that has no ethylenically unsaturated bond is preferable in view of light resistance. Specific examples include acid anhydride-based curing agents such as hexahydrophthalic anhydride, methylhexahydrophthalic anhydride, trialkyltetrahydrophtalic anhydride, and hydrogenated methyl nadic anhydride. Among these acid anhydride-based curing agents, methylhexahydrophthalic anhydride is more preferable.

The curing agents may be used singly or in combination of two or more thereof. The content of the curing agent may be, e.g., a content in which the number of moles of an acid anhydride group or a reactive group equivalent to active hydrogen or the like is 0.4 mol to 2.0 mol based on 1 mol of an epoxy group in the melamine epoxy resin monomer. Preferable is 0.6 mol to 2.0 mol, and further preferable is 0.8 mol to 1.6 mol.

The number of moles of 0.4 mol or more results in good curability and improved reliability. In addition, the number of moles of 2.0 mol or less can inhibit an unreacted curing agent from remaining in a cured product to further improve the moisture resistance of a resultant cured product.

### (Curing Accelerator)

The resin composition preferably contains at least one curing accelerator as well as the curing agent as necessary. As the curing accelerator, a compound usually used as a curing accelerator for an epoxy resin can be used without particular limitation.

Specific examples include imidazoles, quaternary ammonium salts, phosphorus compounds, amines, phosphines, phosphonium salts, bicyclic amidines, and salts thereof. They may be used singly or in combination of two or more thereof.

More specifically, use of imidazoles such as 2-methylimidazole and 2-phenyl-4-imidazole, imidazole salts such as a 2-phenylimidazole-isocyanuric acid adduct, bicyclic amidines such as 1,8-diazabicyclo[5.4.0]undecene-7, carboxylates of bicyclic amidines such as octylate of 1,8-diazabicyclo[5.4.0]undecene-7, and phosphonium salts such as tetraphenyl phosphonium bromide is more preferable since curability is excellent and coloring is suppressed.

The addition amount of the curing accelerator is preferably from 0.1 part by mass to 2 parts by mass based on 100 parts by mass of the melamine epoxy resin monomer.

### (Additive)

The resin composition may contain various additives as necessary. For example, surface regulators such as silane coupling agents, antioxidants, discoloring agents, antidegradants, ultraviolet absorbing agents, mold release agents, plasticizers, diluents, and the like may also be contained.

By containing the surface regulator such as a silane coupling agent, interface adhesive strength between a melamine epoxy resin and an inorganic filler is improved to improve mechanical strength after curing the resin composition.

Examples of the silane coupling agent may include epoxy functional alkoxysilanes such as γ-glycidoxypropyltrimethoxysilane, γ-glycidoxypropylmethyldiethoxysilane, and β-(3,4-epoxycyclohexyl)ethyltrimethoxysilane; amino functional alkoxysilanes such as N-β(aminoethyl)-γ-aminopropyltrimethoxysilane, γ-aminopropyltriethoxysilane, and N-phenyl-γ-aminopropyltrimethoxysilane; and mercapto functional alkoxysilanes such as γ-mercaptopropyltrimethoxysilane. The surface regulator may also be used for surface treatment of the inorganic filler.

The resin composition may contain an antioxidant as necessary. A phenolic, phosphorus-, or sulfur-based antioxidant can be used as the antioxidant, and specific examples of the antioxidant include such antioxidants as described below.

Examples of the phenolic antioxidant include 2,6-di-t-butyl-p-cresol, butylated hydroxyanisole, 2,6-di-t-butyl-p-ethylphenol, stearyl-β-(3,5-di-t-butyl-4-hydroxyphenyl)propionate, 2,2′-methylenebis(4-methyl-6-t-butylphenol), 4,4′-butylilenebis(3-methyl-6-t-butylphenol), 3,9-bis[1,1-dimethyl-2-{β-(3-t-butyl-4-hydroxy-5-methylphenyl]propionyloxy}ethyl]2,4,8,10-tetraoxaspiro[5,5]undecane, 1,1,3-tris(2-methyl-4-hydroxy-5-t-butylphenyl)butane, and 1,3,5-trimethyl-2,4,6-tris(3,5-di-t-butyl-4-hydroxybenzyl)benzene.

Examples of the phosphoric acid-based antioxidant include triphenyl phosphite, diphenylalkyl phosphite, phenyldialkyl phosphite, tri(nonylphenyl) phosphite, trilauryl phosphite, trioctadecyl phosphite, triphenyl phosphite, distearyl pentaerythritol diphosphite, tris(2,4-di-t-butylphenyl)phosphite, diisodecyl pentaerythritol diphosphite, bis(2,4-di-t-butylphenyl)pentaerythritoldiphosphite, tristearyl sorbitol triphosphite, and tetrakis(2,4-di-t-butylphenyl)-4,4′-biphenyldiphosphonate.

In addition, examples of the sulfur-based antioxidant include dilauryl-3,3′-thiodipropionate, dimyristyl-3,3-thiodipropionate, and distearyl-3,3-thiodipropionate.

These antioxidants may be used each singly or in combination of two or more thereof.

The content of the antioxidant is preferably from 0.01 mass % to 10 mass %, and particularly preferably from 0.03 mass % to 5 mass %, in the resin composition. When the content is 0.01 mass % or more, better heat resistance and more effectively suppress discoloration tends to be obtained. In addition, in the case of 10 mass % or less, curing inhibition tends to be suppressed to provide sufficient curability and strength.

### (Method of Producing Resin Composition)

The method of producing the resin composition is not particularly limited, and can be appropriately selected and used from methods usually used as methods for producing an epoxy resin composition. Specifically, for example, the production can be performed by dissolving the melamine epoxy resin monomer in an organic solvent, adding a curing agent and a curing accelerator as necessary, and adding an inorganic filler to the resultant to be mixed.

In addition, the melamine epoxy resin monomer, a curing agent and a curing accelerator which are added as necessary, an inorganic filler, and the like can be thoroughly mixed to become homogeneous by a mixer or the like, thereafter subjected to melt-mix treatment by a heat roll, a kneader, an extruder, or the like, then cooled to solidify, and pulverized to have appropriate sizes to make a molding material of the resin composition.

### (Applications of Resin Composition)

The resin composition is preferably used for applications demanding light resistance as being excellent in light resistance after heat curing. Specifically, the resin composition is preferably used for producing a pre-molded package for a white or blue LED.

A pre-molded package in which the resin composition is used can be produced, for example, by subjecting the resin composition to injection-molding, heating, and pressurization treatment to make a cured product. Heating and pressurization treatment conditions are not particularly limited, and can be appropriately selected depending on the configuration of the resin composition. For example, a temperature of from 150° C. to 200° C., a pressurization condition of from 0.1 MPa to 10 MPa, and from 0.5 minute to 30 minutes are acceptable.

### <Composition for Light Reflection>

The composition for light reflection of the present invention is a cured product of the resin composition. The resin composition can form a cured product excellent in light resistance and optical reflectance by being configured by containing a specific melamine epoxy resin monomer and an inorganic filler and by preferably further containing a curing agent. The details and preferable embodiments of the resin composition are as described above. Especially, the resin composition is preferably configured by containing a white inorganic filler and a curing agent as well as the melamine epoxy resin monomer.

A method of forming the cured product of the resin composition can be appropriately selected from methods usually used for forming epoxy resin compositions depending on the purpose of the composition for light reflection or the like. For example, a method in which the resin composition is subjected to injection-molding or transfer-molding, heating, and pressurization treatment to obtain a cured product, is preferably used. Heating and pressurization treatment conditions are not particularly limited, and can be appropriately selected depending on the configuration of the resin composition, and the heating and pressurization treatment conditions described above can be preferably used.

The composition for light reflection can be used as, for example, pre-molded package for a white or blue LED, or a printed circuit board.

### EXAMPLES

The present invention will be specifically described below by examples, but the present invention is not limited to these examples. Unless otherwise specified, “part(s)” and “%” are based on mass.

### Synthesis Example 1

### [Synthesis of MSE-11-1]

Into a 2,000 ml-separable flask equipped with a stirring machine, a thermometer, a concentrator, and a water separator, 660 g of methylol melamine resin (NIKALAC MS-11: manufactured by Nippon Carbide Industries Co., Inc.), 550 g of epichlorohydrin, 200 g of cyclopentylmethyl ether, 83 g of sodium hydroxide, and 16 g of tetramethylammonium chloride were loaded, vigorously stirred at a reaction temperature of from 45 to 50° C. and under reduced pressure of 10.6 kPa (80 mmHg), to react for 2 hours. Water generated during the reaction and epichlorohydrin were subjected to azeotropy, liquefied in the concentrator, and separated into water and epichlorohydrin in the water separator. The separated water was removed to the outside of a reaction system while the epichlorohydrin was circulated in the reaction system. The reactant was cooled to room temperature and a precipitate was removed by filtration under reduced pressure. 100 g of chloroform was added to the filtrate, washing was performed with 150 g of water three times, and thereafter a solvent was distilling off under reduced pressure to obtain 540 g of a melamine epoxy resin monomer containing a structural unit represented by Formula (I) as a colorless transparent viscous liquid.

The epoxy equivalent of the obtained melamine epoxy resin monomer was determined to be 268 g/eq according to JIS K-7236.

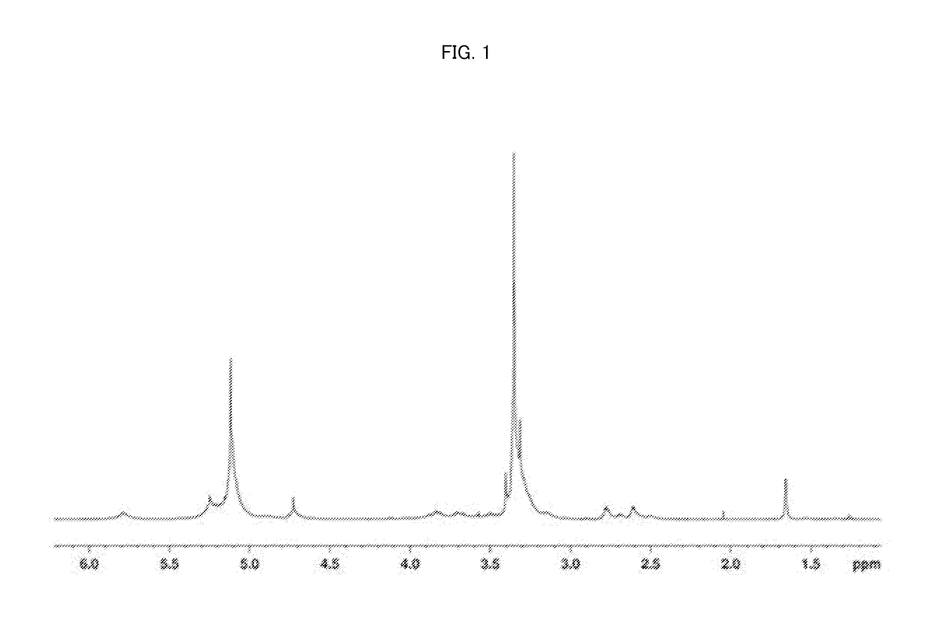

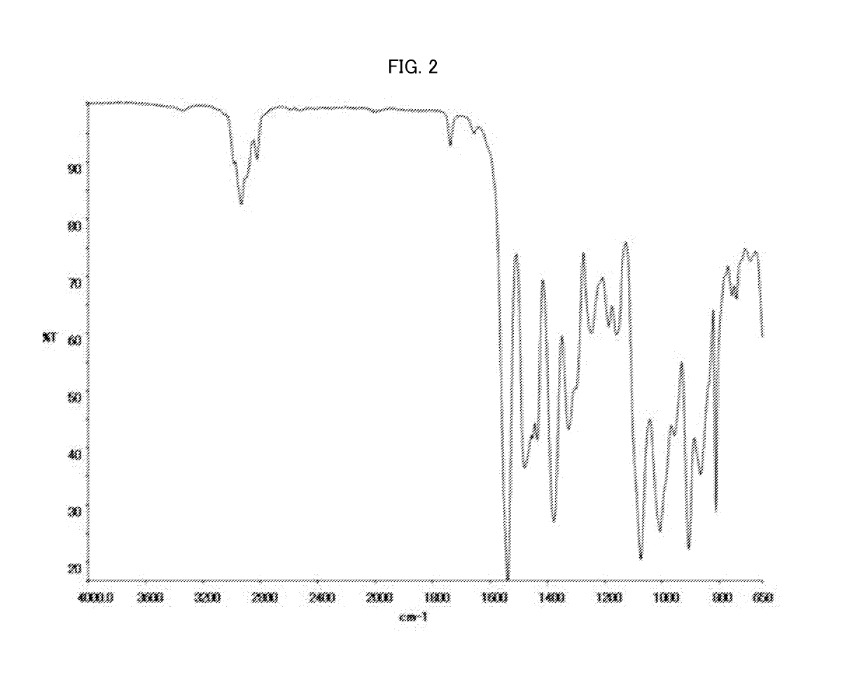

The ¹H-NMR and FT-IR spectra of the obtained melamine epoxy resin monomer in deuterochloroform are indicated in FIG. 1 and FIG. 2, respectively.

### Synthesis Example 2

### [Synthesis of MSE-11-2]

Reaction was carried out by the same method as in Synthesis Example 1 except that reaction time was 1 hour. By distilling off a solvent under reduced pressure, 520 g of a melamine epoxy resin monomer containing a structural unit represented by Formula (I) was obtained as a colorless transparent viscous liquid.

The epoxy equivalent of the obtained melamine epoxy resin monomer was determined to be 300 g/eq according to JIS K-7236.

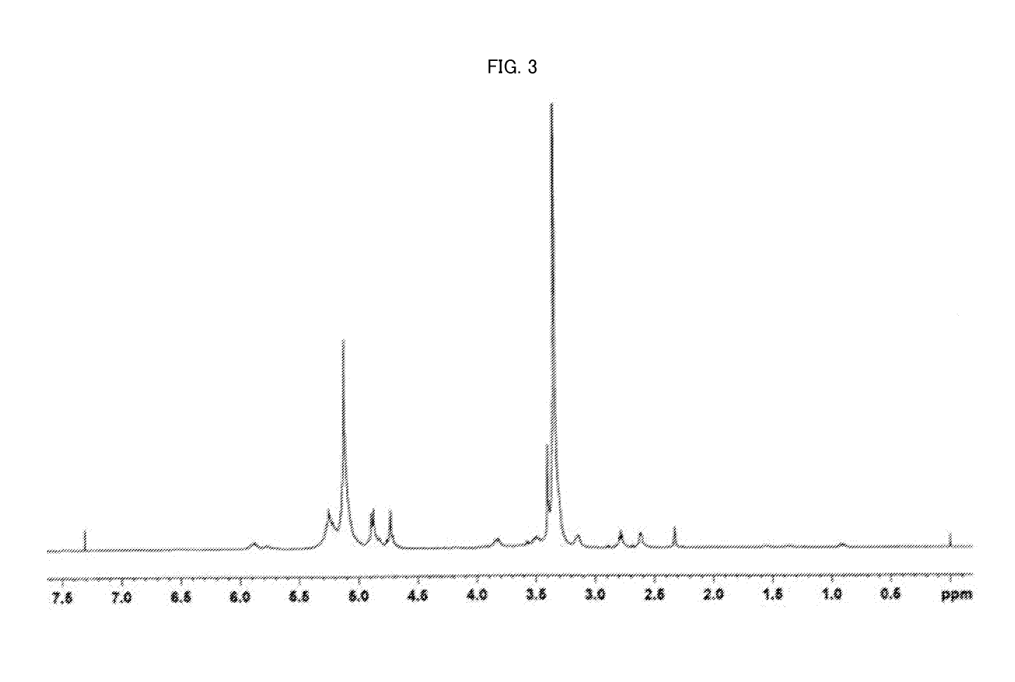

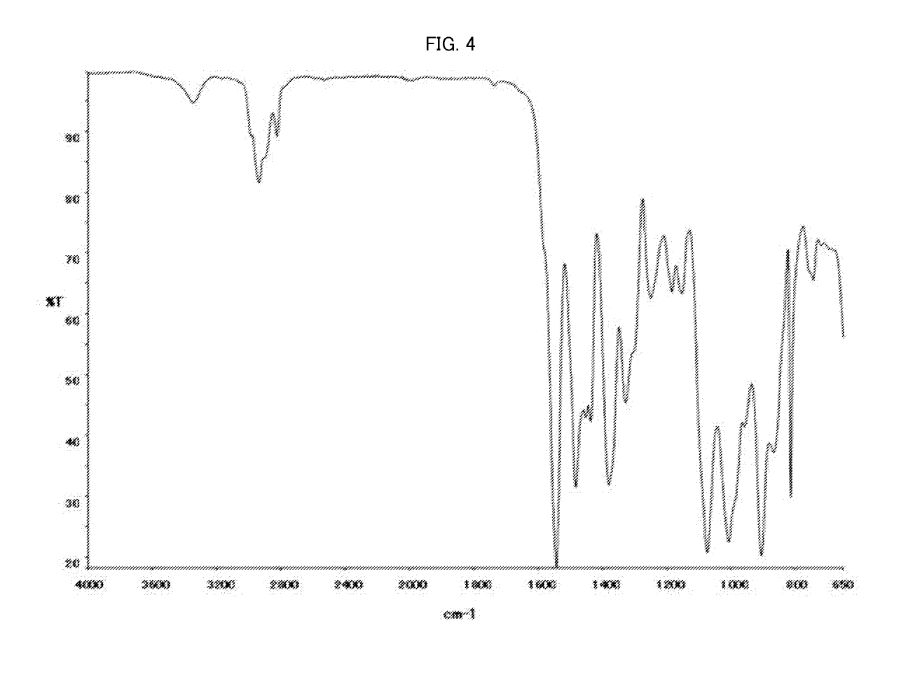

The ¹H-NMR and FT-IR spectra of the obtained melamine epoxy resin monomer in deuterochloroform are indicated in FIG. 3 and FIG. 4, respectively.

### Example 1

### [Preparation of Thermosetting Resin Composition Containing Resin Monomer of Synthesis Example 1 and Titanium Oxide as Inorganic Filler]

In 1.17 parts of ethyl acetate, 0.63 part of the melamine epoxy resin monomer obtained in Synthesis Example 1 was dissolved. To the resultant, 0.35 part of RIKACID MH-700G (trade name, manufactured by New Japan Chemical Co., Ltd., methylhexahydrophthalic anhydride) as a curing agent and 0.02 part of U-CAT SA102 (trade name, manufactured by San-Apro, Ltd., octylate of DBU) as a curing accelerator were added to prepare a resin solution. The resin solution was mixed with 10 g of titanium oxide CR-90-2 (trade name, manufactured by Ishihara Sangyo Kaisha, Ltd., volume-average particle diameter: 0.25 μm) as an inorganic filler and dried at 80° C. for 2 hours to prepare a resin composition as a white powder.

### Example 2

### [Thermosetting Resin Composition Containing Resin Monomer of Synthesis Example 1 and Silica as Inorganic Filler]

A resin composition was prepared in the same manner as in Example 1 except that spherical silica Sciqas 0.7 (trade name, manufactured by Sakai Chemical Industry Co., Ltd., spherical silica, volume-average particle diameter: 0.7 μm) was used as an inorganic filler instead of the titanium oxide CR-90-2.

### Example 3

### [Preparation of Thermosetting Resin Composition Containing Resin of Synthesis Example 1 and Alumina as Inorganic Filler]

A resin composition was prepared in the same manner as in Example 1 except that alumina ALM-41-01 (trade name, manufactured by Sumitomo Chemical Company, Limited, low soda alumina, volume-average particle diameter: 1.5 μm) was used as an inorganic filler instead of the titanium oxide CR-90-2.

### Example 4

### [Preparation of Thermosetting Resin Composition Containing Resin Monomer of Synthesis Example 2 and Alumina as Inorganic Filler]

In 1.17 parts of ethyl acetate, 0.67 part of the melamine epoxy resin monomer obtained in Synthesis Example 2 was dissolved. To the resultant, 0.44 part of DURANATE THA-100 (trade name, manufactured by Asahi Kagaku Chemicals Corp., isocyanurate type isocyanate oligomer) as a curing agent was added to prepare a resin solution. The resin solution was mixed with 10 g of alumina ALM-41-01 (trade name, manufactured by Sumitomo Chemical Company, Limited, low soda alumina, volume-average particle diameter: 1.5 μm) as an inorganic filler and dried at 80° C. for 2 hours to make a white powder to prepare a resin composition.

### Example 5

### [Preparation of Thermosetting Resin Composition Containing Resin Monomer of Synthesis Example 1 and Titanium Oxide, Silica, and Alumina as Inorganic Fillers]

In 1.17 parts of ethyl acetate, 0.63 part of the melamine epoxy resin monomer obtained in Synthesis Example 1 was dissolved. To the result, 0.35 part of RIKACID MH-700G (trade name, manufactured by New Japan Chemical Co., Ltd., methylhexahydrophthalic anhydride) as a curing agent and 0.02 part of HISILICON PX-4ET (trade name, manufactured by Nippon Chemical Industrial Co., Ltd., phosphorus compound) as a curing accelerator were added to prepare a resin solution. The resin solution was mixed with 5 g of titanium oxide CR-90-2 (trade name, manufactured by Ishihara Sangyo Kaisha, Ltd., volume-average particle diameter: 0.25 μm), 3 g of fused silica FB-20D (trade name, manufactured by Denki Kagaku Kogyo Kabushiki Kaisha, volume-average particle diameter: 22 μm), and, in addition, 2 g of low soda alumina ALM-41-01 (trade name, manufactured by Sumitomo Chemical Company, Limited, volume-average particle diameter: 1.5 μm) as inorganic fillers and dried at 80° C. for 2 hours to prepare a resin composition as a white powder.

### <Evaluation>

The physical properties of the resin monomers and the cured products in accordance with Examples and Comparative Examples described above were evaluated by the following procedures. The results are listed in Table 1.

### (1) Epoxy Equivalent

The mass (g) of an epoxy resin containing 1 equivalent of an epoxy group was determined according to JIS K-7236.

### (2) Optical Reflectance

Each thermosetting resin composition prepared in Examples 1 to 3 and Example 5 was subjected to heating and pressurization treatment at a die temperature of 170° C., a pressure of 0.16 MPa, and cure time of 10 minutes to produce a plate test piece having a thickness of 3 mm. Then, the optical reflectance of each test piece at a wavelength of 460 nm was measured using a spectrophotometer U-4000 type (manufactured by Hitachi, Ltd.). The reflectance of each test piece is a relative value assuming that barium sulfate has a reflectance of 100%.

In addition, the thermosetting resin composition prepared in Example 4 was subjected to heating and pressurization treatment at a die temperature of 200° C., and a pressure of 0.16 MPa, and cure time of 10 minutes to produce a plate test piece having a thickness of 3 mm, and the plate test piece was subjected to the same test.

### (3) Heat Resistance

Each thermosetting resin composition prepared in Examples 1 to 3 and Example 5 was subjected to heating and pressurization treatment at a die temperature of 170° C., a pressure of 0.16 MPa, and cure time of 10 minutes to produce a plate test piece having a thickness of 3 mm. Then, the optical reflectance of each test piece at a wavelength of 460 nm was measured and evaluated in the same manner as described above after left unattended at 170° C. for 2 hours.

In addition, the same test was conducted by subjecting a plate test piece having a thickness of 3 mm produced by subjecting the thermosetting resin composition prepared in Example 4 to heating and pressurization treatment at a die temperature of 200° C., a pressure of 0.16 MPa, and cure time of 10 minutes.

### (4) Light Resistance

Each thermosetting resin composition prepared in Examples 1 to 3 and Example 5 was subjected to heating and pressurization treatment at a die temperature of 170° C., a pressure of 0.16 MPa, and cure time of 10 minutes to produce a plate test piece having a thickness of 3 mm. Then, a test was conducted under conditions of an irradiation intensity of 850 W/cm², a temperature of 83° C., a humidity of 20 RH %, and no dewing for 100 hours using METAL WEATHER KW-RSTP-A (manufactured by Daypla Wintes Co., Ltd.), followed by measuring and evaluating the optical reflectance of each test piece in the same manner as described above.

In addition, the same test was conducted by subjecting a plate test piece having a thickness of 3 mm produced by subjecting the thermosetting resin composition prepared in Example 4 to heating and pressurization treatment at a die temperature of 200° C., a pressure of 0.16 MPa, and cure time of 10 minutes.

TABLE 1

| | | Example 1 | Example 2 | Example 3 | Example 4 | Example 5 |
|---|---|---|---|---|---|---|
| Melamine epoxy resin | Synthesis Example 1 | 0.63 | 0.63 | 0.63 | — | 0.63 |
| monomer | (part) | | | | | |
| | Synthesis Example 2 | — | — | — | 0.67 | — |
| | (part) | | | | | |
| Curing agent | MH-700G (part) | 0.35 | 0.35 | 0.35 | — | 0.35 |
| | THA-100 (part) | — | — | — | 0.44 | — |
| Curing accelerator | U-CAT SA102 (part) | 0.02 | 0.02 | 0.02 | — | — |
| | PX-4ET (part) | — | — | — | — | 0.02 |
| Inorganic filler | CR-90-2 (part(s)) | 10 | — | — | — | 5 |
| | Sciqas (part(s)) | — | 10 | — | — | — |
| | ALM-41-01 (part(s)) | — | — | 10 | 10 | 2 |
| | FB-20D (part(s)) | — | — | 10 | 10 | 3 |

| | | | | | |
|---|---|---|---|---|---|
| Optical reflectance (%) | 99.3 | 100.0 | 95.3 | 93.8 | 99.2 |
| Optical reflectance (%) after heat resistance test | 98.9 | 99.1 | 95.9 | 94.2 | 98.6 |
| Optical reflectance (%) after light resistance test | 99.0 | 100.0 | 97.9 | 95.3 | 99.1 |

Table 1 reveals that the resin composition of the present invention is capable of forming a cured product that has a high optical reflectance and is excellent in light resistance and heat resistance by heat curing. It is noted that “-” in Table 1 indicates unblending.

In addition, the resin composition of Example 5 was confirmed to have improved flowability compared with the resin compositions in the other examples.

The disclosure of Japanese Patent Application No. 2010-219953 is incorporated herein by reference in its entirety.

All literatures, patent applications, and technical standards described in this specification are incorporated herein by reference to the same extent as if each individual literature, patent application and technical standard were specifically and individually indicated as being incorporated by reference.

1. A melamine epoxy resin monomer comprising: a glycidyl group; and a structural unit having a melamine residue and being represented by the following Formula (I): 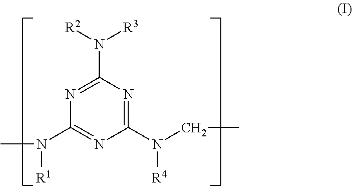
wherein in Formula (I), each of R¹ to R⁴ independently represents a hydrogen atom, a group represented by R⁵OCH₂—, or a group derived from a melamine derivative and represented by the following Formula (II); and R⁵ represents a hydrogen atom, an alkyl group having 1 to 4 carbon atoms, or a glycidyl group: 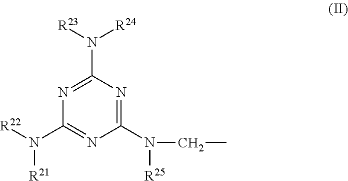
wherein in Formula (II), each of R²¹ to R²⁵ independently represents a hydrogen atom, a group represented by R²⁶OCH₂—, or a group derived from a melamine derivative and represented by Formula (II); and R²⁶ represents a hydrogen atom, an alkyl group having 1 to 4 carbon atoms, or a glycidyl group.

2. A melamine epoxy resin monomer comprising a glycidyl group and a melamine residue and being represented by the following Formula (III): 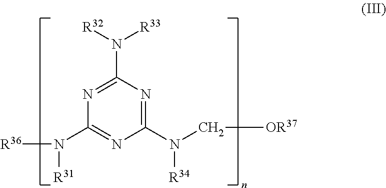
wherein in Formula (III), each of R³¹ to R³⁴ independently represents a hydrogen atom, a group represented by R³⁵OCH₂—, or a group derived from a melamine derivative and represented by the following Formula (II); R³⁶ represents a hydrogen atom or a group represented by R³⁸OCH₂—; each of R³⁵, R³⁷, and R³⁸ independently represents a hydrogen atom, an alkyl group having 1 to 4 carbon atoms, or a glycidyl group; and n represents an integer from 1 to 8: 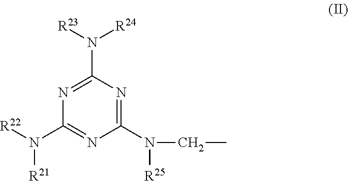
wherein in Formula (II), each of R²¹ to R²⁵ independently represents a hydrogen atom, a group represented by R²⁶OCH₂—, or a group derived from a melamine derivative and represented by Formula (II); and R²⁶ represents a hydrogen atom, an alkyl group having 1 to 4 carbon atoms, or a glycidyl group.

3. The melamine epoxy resin monomer according to claim 1, wherein the number of the contained glycidyl groups is 2 or more.

4. The melamine epoxy resin monomer according to claim 1, wherein the number of the contained melamine residues is 8 or less.

5. A resin composition comprising: the melamine epoxy resin monomer according to claim 1; and an inorganic filler.

6. The resin composition according to claim 5, further comprising a curing agent.

7. A composition for light reflection, which is a cured product of the resin composition according to claim 5.

8. The melamine epoxy resin monomer according to claim 2, wherein the number of the contained glycidyl groups is 2 or more.

9. The melamine epoxy resin monomer according to claim 2, wherein the number of the contained melamine residues is 8 or less.

10. A resin composition comprising: the melamine epoxy resin monomer according to claim 2; and an inorganic filler.

11. The resin composition according to claim 10, further comprising a curing agent.

12. A composition for light reflection, which is a cured product of the resin composition according to claim 11.

13. The melamine epoxy resin monomer according to claim 1, wherein:
the number of the contained glycidyl groups is 2 or more; and
the number of the contained melamine residues is 8 or less.

14. A resin composition comprising: the melamine epoxy resin monomer according to claim 13; and an inorganic filler.

15. The resin composition according to claim 14, further comprising a curing agent.

16. A composition for light reflection, which is a cured product of the resin composition according to claim 15.

17. The melamine epoxy resin monomer according to claim 2, wherein:
the number of the contained glycidyl groups is 2 or more; and
the number of the contained melamine residues is 8 or less.

18. A resin composition comprising: the melamine epoxy resin monomer according to claim 17; and an inorganic filler.

19. The resin composition according to claim 18, further comprising a curing agent.

20. A composition for light reflection, which is a cured product of the resin composition according to claim 19.
